SEQUENCE LISTING (44 sequences)

SEQ ID NO: 1 - protein, 10 aa, synthetic construct
FSTAADGQTQ

SEQ ID NO: 2 - protein, 13 aa, synthetic construct
GGGFSTAADGQTQ

SEQ ID NO: 3 - protein, 7 aa, synthetic construct
DTETKME

SEQ ID NO: 4 - protein, 11 aa, synthetic construct
RSIYYLGIGPT

SEQ ID NO: 5 - protein, 11 aa, synthetic construct
YARVRRRGPRR

SEQ ID NO: 6 - protein, 27 aa, synthetic construct
GWTLNSAGYLLGKINLKALAALAKKIL

SEQ ID NO: 7 - protein, 21 aa, synthetic construct
KETWWETWWTEWSQPKKKRKV

SEQ ID NO: 8 - protein, 16 aa, synthetic construct
LLIILRRRIRKQAHAH

SEQ ID NO: 9 - protein, 22 aa, synthetic construct
MVTVLFRRLRIRRACGPPRVRV

SEQ ID NO: 10 - protein, 21 aa, synthetic construct
AGYLLGKINLKALAALAKKIL

SEQ ID NO: 11 - protein, 34 aa, synthetic construct
DAATATRGRSAASRPTERPRAPARSASRPRRPVE

SEQ ID NO: 12 - protein, 20 aa, synthetic construct
TRSSRAGLQFPVGRVHRLLR

SEQ ID NO: 13 - protein, 30 aa, synthetic construct
WEAKLAKALAKALAKHLAKALAKALKACEA

SEQ ID NO: 14 - protein, 35 aa, synthetic construct
KKKKKKGGFLGFWRGENGRKTRSAYERMCNILKGK

SEQ ID NO: 15 - protein, 42 aa, synthetic construct
YKQCHKKGGHCFPKEKICLPPSSDFGKMDCRWRWKCCKKGSG

SEQ ID NO: 16 - protein, 9 aa, synthetic construct
CRGDKGPDC

SEQ ID NO: 17 - protein, 24 aa, synthetic construct
KIKKVKKKGRKGSKIKKVKKKGRK

SEQ ID NO: 18 - protein, 24 aa, synthetic construct
KIKKRKKKGRKGSKIKKVKKKGRK

SEQ ID NO: 19 - protein, 24 aa, synthetic construct
KIKKRKKKGRKGSKIKKRKKKGRK

SEQ ID NO: 20 - protein, 9 aa, synthetic construct
RRRRRRRRR

SEQ ID NO: 21 - protein, 21 aa, synthetic construct
YARVRRRGPRRFSTAADGQTQ

SEQ ID NO: 22 - protein, 24 aa, synthetic construct
YARVRRRGPRRGGGFSTAADGQTQ

SEQ ID NO: 23 - protein, 18 aa, synthetic construct
YARVRRRGPRRDTETKME

SEQ ID NO: 24 - protein, 22 aa, synthetic construct
YARVRRRGPRRRSIYYLGIGPT

SEQ ID NO: 25 - protein, 347 aa, synthetic construct
MPLIYWFSRRMTLWGSISFNLAVFINIIIAFFYPYMEGASTGVLDSPLISLLFWILICFSIAALFTKRYSIRPLIVALILRSIYYLGIGPTLNILGALNLTNKIVFVVSFVGNRGTFIRGYKAMVMDMEFLYHVGYILTSVLGLFAHELFYSILLFDLIYREETLFNVIKSVTRNGRSILLTALLALILVYLFSIVGFLFLKDDFILEVDRLPNNHSTASPLGMPHGAAAFVDTCSGDKMDCVSGLSVPEVLEEDRELDSTERACDTLLMCIVTVMNHGLRNGGGVGDILRKPSKDESLFPARVVYDLLFFFIVIIIVLNLIFGVIIDTFADLRSEKQKKEEILKTT

SEQ ID NO: 26 - protein, 11 aa, synthetic construct
RSIYYLGIGPT

SEQ ID NO: 27 - protein, 159 aa, synthetic construct
DVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKME

SEQ ID NO: 28 - protein, 10 aa, synthetic construct
FSTAADGQTQ

SEQ ID NO: 29 - protein, 7 aa, synthetic construct
DTETKME

SEQ ID NO: 30 - protein, 38 aa, synthetic construct
GWTLNSAGYLLGKINLKALAALAKKILRSIYYLGIGPT

SEQ ID NO: 31 - protein, 32 aa, synthetic construct
KETWWETWWTEWSQPKKKRKVRSIYYLGIGPT

SEQ ID NO: 32 - protein, 27 aa, synthetic construct
LLIILRRRIRKQAHAHRSIYYLGIGPT

SEQ ID NO: 33 - protein, 33 aa, synthetic construct
MVTVLFRRLRIRRACGPPRVRVRSIYYLGIGPT

SEQ ID NO: 34 - protein, 32 aa, synthetic construct
AGYLLGKINLKALAALAKKILRSIYYLGIGPT

SEQ ID NO: 35 - protein, 45 aa, synthetic construct
DAATATRGRSAASRPTERPRAPARSASRPRRPVERSIYYLGIGPT

SEQ ID NO: 36 - protein, 31 aa, synthetic construct
TRSSRAGLQFPVGRVHRLLRRSIYYLGIGPT

SEQ ID NO: 37 - protein, 41 aa, synthetic construct
WEAKLAKALAKALAKHLAKALAKALKACEARSIYYLGIGPT

SEQ ID NO: 38 - protein, 46 aa, synthetic construct
KKKKKKGGFLGFWRGENGRKTRSAYERMCNILKGKRSIYYLGIGPT

SEQ ID NO: 39 - protein, 53 aa, synthetic construct
YKQCHKKGGHCFPKEKICLPPSSDFGKMDCRWRWKCCKKGSGRSIYYLGIGPT

SEQ ID NO: 40 - protein, 20 aa, synthetic construct
CRGDKGPDCRSIYYLGIGPT

SEQ ID NO: 41 - protein, 35 aa, synthetic construct
KIKKVKKKGRKGSKIKKVKKKGRKRSIYYLGIGPT

SEQ ID NO: 42 - protein, 35 aa, synthetic construct
KIKKRKKKGRKGSKIKKVKKKGRKRSIYYLGIGPT

SEQ ID NO: 43 - protein, 35 aa, synthetic construct
KIKKRKKKGRKGSKIKKRKKKGRKRSIYYLGIGPT

SEQ ID NO: 44 - protein, 20 aa, synthetic construct
RRRRRRRRRRSIYYLGIGPT

(19) United States
(12) Patent Application Publication
(10) Pub. No.: US 2025/0171496 A1
(43) Pub. Date: May 29, 2025

(54) FUSION PEPTIDE FOR REGULATING ACTIVATION OF MAM AND USE THEREOF

(71) Applicant: UIF (University Industry Foundation), Yonsei University, Seoul (KR)

(72) Inventors: Ho Jeong KWON, Seoul (KR); Jung Min HA, Gyeonggi-do (KR)

(73) Assignee: UIF (University Industry Foundation), Yonsei University, Seoul (KR)

(21) Appl. No.: 18/958,856

(22) Filed: Nov. 25, 2024

(30) Foreign Application Priority Data
Nov. 28, 2023 (KR) 10-2023-0167828
Nov. 13, 2024 (KR) 10-2024-0161195

(51) Int. Cl. C07K 7/06; A61K 38/00; A61P 9/10; C07K 14/47

(52) U.S. Cl. CPC C07K 7/06; A61P 9/10; C07K 14/4702; A61K 38/00; C07K 2319/00

(57) ABSTRACT

The present disclosure relates to a peptide for inhibiting PPI between IP₃R and GRP75 in MAM, and a composition for preventing, alleviating or treating a disease related to autophagy disorder containing the same as an active ingredient.

The features and advantages of the present disclosure may be summarized as follows:
(a) The present disclosure provides a peptide capable of inducing intracellular autophagy by inhibiting the interaction between the endoplasmic reticulum and mitochondria, more specifically the PPI between IP₃R and GRP75 in MAM, by regulating the activity of MAM.
(b) The peptide of the present disclosure can be usefully applied to alleviate or treat a disease related to autophagy disorder, especially a disease caused by abnormal protein accumulation induced by reduced intracellular autophagy or a degenerative disease.

### BACKGROUND

### 1. Field

The present disclosure relates to a fusion peptide for PPI inhibition between IP₃R and GRP75 in MAM, and a composition for preventing, alleviating or treating a disease related to autophagy disorder containing the same as an active ingredient. The fusion peptide increases the distance between the endoplasmic reticulum and mitochondria, thereby separating them and correspondingly inhibiting the interaction between the endoplasmic reticulum and mitochondria, thereby increasing intracellular autophagic activity. Therefore, it can be usefully used to alleviate or treat diseases associated with autophagy dysfunction.

### 2. Description of the Related Art

There are many protein complexes at the contact sites of intracellular organelles, and they communicate with each other through interactions. The function of the cellular organelles is controlled by the regulation of these protein complexes. Among them, the protein complexes present in MAMs (mitochondria-associated membranes), which are the contact points between mitochondria and the endoplasmic reticulum, regulate intracellular energy metabolism, calcium transfer, autophagy, lipid homeostasis, etc. In particular, IP₃R, which is a calcium release channel located in the endoplasmic reticulum membrane, and VDAC1, located in the outer mitochondrial membrane, transfer calcium from the endoplasmic reticulum to the mitochondria, and they are physically linked by GRP75, which is a chaperone protein located in the cytoplasm. Because intracellular calcium transport plays a key role in maintaining of cellular homeostasis, signal transduction, etc., research on the IP₃R-GRP75-VDAC1 complex are increasing.

Meanwhile, a recent study reported that autophagy is induced as the distance between the endoplasmic reticulum and mitochondria increases. Autophagy is the process of degrading dysfunctional organelles or proteins for use as an energy source for a cell. It is activated in situations of cellular stress. Therefore, abnormalities in autophagic activity can lead to metabolic diseases, immune diseases, vascular diseases, etc.

Accordingly, the development of a peptide that can induce autophagy by inhibiting the interaction between the endoplasmic reticulum and mitochondria, which are intracellular organelles, more specifically, protein-protein interaction (PPI) between IP₃R (inositol 1,4,5-trisphosphate (IP₃) receptor) and GRP75 (glucose-related protein 75) in MAMs (mitochondria-associated membranes) by regulating the activity of the MAMs has recently attracted attention.

Throughout this specification, reference is made to and citations are provided for a number of literatures and patent documents. The disclosures of the cited literature and patent documents are incorporated in this specification by reference in their entirety in order to more clearly explain the level of the technical field to which the present disclosure belongs and the contents of the present disclosure.

### REFERENCES OF RELATED ART

### Non-Patent Documents

(Non-patent document 1) Filadi, R., Theurey, P. & Pizzo, P. The endoplasmic reticulum-mitochondria coupling in health and disease: Molecules, functions and significance. Cell Calcium 62, 1-15 (2017).
(Non-patent document 2) Xu, L., Wang, X. & Tong, C. Endoplasmic Reticulum-Mitochondria Contact Sites and Neurodegeneration. Front. Cell Dev. Biol. 8, (2020).
(Non-patent document 3) Wilson, E. L. & Metzakopian, E. ER-mitochondria contact sites in neurodegeneration: genetic screening approaches to investigate novel disease mechanisms. Cell Death Differ. 2020 286 28, 1804-1821 (2020).
(Non-patent document 4) Mabonga, L. & Kappo, A. P., Protein-protein interaction modulators: advances, successes and remaining challenges. Biophys. Rev. 2019 114 11, 559-581 (2019).
(Non-patent document 5) Gomez-Suaga, P. et al. The ER-Mitochondria Tethering Complex VAPB-PTPIP51 Regulates Autophagy. Curr. Biol. 27, 371-385 (2017).
(Non-patent document 6) Mizushima, N. & Komatsu, M. Autophagy: Renovation of cells and tissues. Cell 147, 728-741 (2011).
(Non-patent document 7) Ichimiya, T. et al. Autophagy and Autophagy-Related Diseases: A Review. Int. J. Mol. Sci. 21, 1-21 (2020).
(Non-patent document 8) Wang, K. et al. Peptide-based PROTAC degrader of FOXM1 suppresses cancer and decreases GLUT1 and PD-L1 expression. J. Exp. Clin. Cancer Res. 41, 1-15 (2022).
(Non-patent document 9) Choi, J. M. et al. Transduction of the cytoplasmic domain of CTLA-4 inhibits TcR-specific activation signals and prevents collagen-induced arthritis. Proc. Natl. Acad. Sci. U.S.A 105, 19875-19880 (2008).
(Non-patent document 10) Choi, J. M. et al. Intranasal delivery of the cytoplasmic domain of CTLA-4 using a novel protein transduction domain prevents allergic inflammation. Nat. Med. 12, 574-579 (2006).
(Non-patent document 11) Frankel, A D & Pabo, C. O. Cellular uptake of the tat protein from human immunodeficiency virus. Cell 55, 1189-1193 (1988).
(Non-patent document 12) Cal, T. & Brini, M. Quantification of organelle contact sites by split-GFP-based contact site sensors (SPLICS) in living cells. Nat. Protoc. 16, 5287-5308 (2021).
(Non-patent document 13) Miki, Y., Iwabuchi, E., Ono, K., Sasano, H. & Ito, K. Exploring Protein-Protein Interaction in the Study of Hormone-Dependent Cancers. Int. J. Mol. Sci. 2018, Vol. 19, Page 3173 19, 3173 (2018).
(Non-patent document 14) Razani, B. et al. Autophagy links inflammasomes to atherosclerotic progression. Cell Metab. 15, 534-544 (2012).

### SUMMARY

The present disclosure is directed to providing a fusion peptide for regulating MAM activity of the endoplasmic reticulum and mitochondria, specifically a fusion peptide for inhibiting the interaction between the endoplasmic reticulum and mitochondria, and more specifically a fusion peptide for inhibiting PPI between IP₃R and GRP75 in MAM.

The present disclosure is also directed to providing a pharmaceutical composition for preventing or treating a disease related to autophagy disorder, which contains the fusion peptide as an active ingredient.

The present disclosure is also directed to providing a food composition for preventing or alleviating a disease related to autophagy disorder, which contains the fusion peptide as an active ingredient.

The present disclosure is also directed to providing a composition for activating autophagy in vitro, which contains the fusion peptide as an active ingredient.

The present disclosure is also directed to providing a composition for inhibiting the interaction between the endoplasmic reticulum and mitochondria in vitro, which contains the fusion peptide as an active ingredient.

The present disclosure provides a fusion peptide for inhibiting protein-protein interaction (PPI) between IP₃R (1,4,5-trisphosphate (IP₃) receptor) and GRP75 (Glucose-related protein 75) in MAM (mitochondria-associated membranes), which contains a peptide represented by the amino acid sequence of SEQ ID NO: 4 and a cell-penetrating peptide (CPP).

In an exemplary embodiment of the present disclosure, the cell-penetrating peptide may be represented by any amino acid sequence selected from SEQ ID NOS: 5 to 20.

In an exemplary embodiment of the present disclosure, the fusion peptide may be represented by any amino acid sequence selected from SEQ ID NO: 24 and SEQ ID NOS: 30 to 44.

In an exemplary embodiment of the present disclosure, the fusion peptide may have one or more characteristics selected from 1) to 4):
1) inhibition of calcium transport from the endoplasmic reticulum to mitochondria;
2) increase in lysosomal activity;
3) increase in autophagic activity; and
4) increased distance between the endoplasmic reticulum and mitochondria.

The present disclosure also provides a nucleic acid molecule encoding the fusion peptide.

The present disclosure also provides an expression vector containing the nucleic acid molecule.

The present disclosure also provides a nucleic acid molecule encoding the fusion peptide.

The present disclosure also provides a host cell transformed with the expression vector.

The present disclosure also provides a pharmaceutical composition for preventing or treating a disease related to autophagy disorder, which contains the fusion peptide, the nucleic acid molecule, the expression vector or the host cell as an active ingredient.

In an exemplary embodiment of the present disclosure, the fusion peptide can inhibit protein-protein interaction (PPI) between IP₃R and GRP75 in MAMs (mitochondria-associated membranes).

In an exemplary embodiment of the present disclosure, the disease related to autophagy disorder may be a disease caused by abnormal protein accumulation or a degenerative disease.

In an exemplary embodiment of the present disclosure, the disease caused by abnormal protein accumulation or the degenerative disease may be any one selected from arteriosclerosis, pulmonary hypertension, Alzheimer's disease, Parkinson's disease, type 2 diabetes, amyotrophic lateral sclerosis, dialysis-related amyloidosis, cystic fibrosis, sickle cell anemia, Huntington's disease, Creutzfeldt-Jakob disease, Lewy body dementia, inclusion body myositis, cerebral amyloid angiopathy, traumatic brain injury, frontotemporal dementia, progressive supranuclear palsy, corticobasal degeneration, Pick's disease, and argyrophilic grain disease, induced by reduced intracellular autophagy.

In an exemplary embodiment of the present disclosure, the arteriosclerosis may be atherosclerosis.

In an exemplary embodiment of the present disclosure, the composition may reduce intracellular lipids by inducing autophagy.

The present disclosure also provides a food composition for preventing or alleviating a disease related to autophagy disorder, which contains the fusion peptide, the nucleic acid molecule, the expression vector or the host cell as an active ingredient.

The present disclosure also provides a composition for activating autophagy in vitro, which contains the fusion peptide, the nucleic acid molecule, the expression vector or the host cell as an active ingredient.

The present disclosure also provides a composition for inhibiting the interaction of the endoplasmic reticulum and mitochondria in vitro, which contains the fusion peptide, the nucleic acid molecule, the expression vector or the host cell as an active ingredient.

The features and advantages of the present disclosure may be summarized as follows:
(a) The present disclosure provides a fusion peptide that can induce intracellular autophagy by inhibiting the interaction between the endoplasmic reticulum and mitochondria, more specifically, PPI between IP₃R and GRP75 in MAM, by regulating the activity of MAM.
(b) The fusion peptide of the present disclosure can be usefully used to alleviate or treat a disease related to autophagy disorder, specifically, a disease caused by abnormal protein accumulation induced by reduced intracellular autophagy or a degenerative disease.

### BRIEF DESCRIPTION OF THE DRAWINGS

FIGS. 1A and 1B show a result of validating MAM regulatory activity of fusion peptides. After transfecting Hela cells with a SPLICS vector, MAM proximity was increased by treating them with 10 mM ethanol (EtOH). Fluorescence images were taken after treating the cells with four types of fusion peptides at 20 μM for 1 hour. Fluorescence signals were attenuated when the fusion peptides were treated as compared to a control group (DW) (FIG. 1A). Hela cells were treated with four types of fusion peptides at 20 μM for 1 hour. After inducing calcium release from the endoplasmic reticulum by treating the cells with 200 μM histamine, and mitochondrial calcium level was measured. Mitochondrial calcium influx was reduced when the fusion peptide 4 was treated as compared to the fusion peptides 1 to 3 (FIG. 1B).

FIGS. 2A and 2B show a result of validating MAM regulatory activity of the fusion peptide 4. Hela cells were treated with the fusion peptide 4 at 20 μM for 1 hour. Fluorescence images were obtained by performing PLA. Fluorescence was attenuated when the cells were treated with fusion peptide as compared to a control (DW) group. The white arrows indicate red fluorescence dots (FIG. 2A). The positions of peptide sequences are indicated in the structures of human IP₃R, GRP75 and VDAC1 obtained from AlphaFold (FIG. 2B).

FIGS. 3A and 3B show a result of validating intracellular permeability and determining the appropriate concentration of the fusion peptide 4. HUVEC cells were treated with the fusion peptide 4 labeled with FITC at concentrations of 5 μM (top) and 10 μM (bottom) for 3 hours, and then fluorescence was observed. Concentration-dependent intracellular permeability was observed (FIG. 3A). HUVEC cells were treated with the fusion peptide 4 at concentrations of 0 to 300 μM, and cell proliferation was observed for up to 72 hours by MTT assay (FIG. 3B).

FIGS. 4A and 4B show TFEB nuclear translocation and lysosomal activity depending on treatment with the fusion peptide 4. After treating Hela cells with the fusion peptide 4 at 20 and 50 μM, the location of TFEB was investigated for 0 to 12 hours. After treatment of Hela cells with fusion peptide 4 at 20 and 50 μM, the location of TFEB was confirmed for 0 to 12 hours. TFEB nuclear translocation occurred over time. Representative image shows the result of treatment with 20 μM fusion peptide 4. A result of quantifying the colocalization of TFEB (green) and the nucleus (blue) is shown in the graphs (FIG. 4A). Lysosomal activity was increased after the HUVEC cells were treated with the fusion peptide 4 at a concentration of 100 μM for 12 and 24 hours (FIG. 4B).

FIG. 5 shows a result of inducing autophagic flux by treating with the fusion peptide 4. HUVEC cells were treated with the fusion peptide 4 at 200 μM. The conversion of LC3B I to LC3B II, which was inhibited by oxLDL (50 μg/mL), was restored by the treatment with the fusion peptide 4.

FIGS. 6A and 6B show the effect of alleviating atherosclerosis of the fusion peptide 4. Atherosclerotic mitigating effect of fusion peptide 4. FIG. 6A shows triglyceride (TG) and total cholesterol levels in the serum of a control group (vehicle), a test group (peptide), and a positive control group (CTS, cryptotanshinone). FIG. 6B shows a result of isolating the arteries of mice and observing neutral lipids by staining with Oil Red O.

### DETAILED DESCRIPTION

Hereinafter, exemplary embodiments of the present disclosure will be described in detail with reference to the attached drawings. However, the following exemplary embodiments are provided only as examples of the present disclosure, and if it is determined that a detailed description of a technology or configuration well known to those skilled in the art may unnecessarily obscure the gist of the present disclosure, the detailed description may be omitted, and the present disclosure is not limited thereby. The present disclosure can be changed and modified variously within the scope of the appended claims and equivalents interpreted therefrom.

All technical terms used in the present disclosure, unless otherwise defined, have the same meaning as commonly understood by those skilled in the art in the relevant field of the present disclosure. In addition, although preferred methods and samples are described in this specification, similar or equivalent methods and samples are also included in the scope of the present disclosure. The contents of all publications described in the present specification are incorporated herein by reference.

Throughout this specification, the conventional one-letter and three-letter codes for naturally occurring amino acids are used, as well as the generally accepted three-letter codes for other amino acids, such as Aib (α-aminoisobutyric acid), Sar (N-methylglycine), etc. Additionally, the amino acids referred to in the present disclosure as abbreviations are described according to the IUPAC-IUB nomenclature as follows: alanine: A, arginine: R, asparagine: N, aspartic acid: D, cysteine: C, glutamic acid: E, glutamine: Q, glycine: G, histidine: H, isoleucine: I, leucine: L, lysine: K, methionine: M, phenylalanine: F, proline: P, serine: S, threonine: T, tryptophan: W, tyrosine: Y and valine: V.

The present inventors have made extensive efforts to discover a novel fusion peptide that can induce intracellular autophagy by inhibiting the interaction between the endoplasmic reticulum and mitochondria, which are intracellular organelles, more specifically, PPI between IP₃R and GRP75 in MAM, by regulating the activity of MAM. As a result, they have predicted the binding domain of IP₃R and GRP75 through analysis of structural information and bioinformatics of a protein complex consisting of IP₃R, which is an endoplasmic reticulum calcium release channel, VDAC1, which is a voltage-dependent anion channel in mitochondria, and GRP75, which acts as a bridge between the two molecules. Then, they have prepared a novel fusion peptide in which a part of the transmembrane domain present in IP₃R and a membrane-permeable sequence are linked to regulate the same. In addition, they have completed the present disclosure by confirming that the fusion peptide can be used to prevent, alleviate or treat a disease related to autophagy disorder since it binds to the transmembrane domain of IP₃R to inhibit the interaction between IP₃R and GRP75 and induce intracellular autophagy.

In this specification, the term “autophagy” refers to a mechanism related to cell survival and death. Unlike apoptosis or necrosis in which the entire cell dies, it involves processes in which cell organelles are continuously degraded and regenerated. Through this process, cellular damage is restored, intracellular structures are rebuilt, and intracellular nutrients, energy necessary for survival, and proteins are resupplied, allowing the cell to adapt to stressful environments and regain homeostasis. Autophagy is an essential and evolutionarily conserved pathway that plays a critical role in maintaining cellular function and viability in response to stress. Recently, many studies have been reported to reduce oxidative stress caused by ototoxicity by utilizing autophagy.

The present disclosure will now be described in detail.

An aspect of the present disclosure relates to a fusion peptide for inhibiting protein-protein interaction (PPI) between IP₃R (inositol 1,4,5-trisphosphate (IP₃) receptor) and GRP75 (glucose-related protein 75) in MAMs (mitochondria-associated membranes), which contains a peptide represented by the amino acid sequence of SEQ ID NO: 4 and a cell-penetrating peptide (CPP).

The peptide and/or fusion peptide may be subject to insertion, substitution, or deletion of other amino acids within a range that does not significantly impair the purpose, function, and stability of the present disclosure, and this also falls within the scope of the present disclosure.

In a specific exemplary embodiment, the cell permeability of the fusion peptide of the present disclosure can be further improve by linking a cell-penetrating peptide to the N-terminus of the peptide represented by the amino acid sequence of SEQ ID NO: 4, and such a fusion peptide may also be referred to as a ‘fused peptide’ or ‘fusion protein’.

The ‘fusion peptide’, ‘fused peptide’ or ‘fusion protein’ contains the peptide represented by the amino acid sequence of SEQ ID NO: 4 and a cell-penetrating peptide, and means a covalent complex formed by genetic fusion or chemical bonding thereof.

And, the term “genetic fusion” refers to a linear, covalent linkage formed through genetic expression of a DNA sequence encoding a protein.

In a specific exemplary embodiment, the cell-penetrating peptide may be represented by any amino acid sequence selected from SEQ ID NOS: 5 to 20. Additionally, the cell-penetrating peptide may be any one selected from HIV-1 Tat (47-57), D-amino acid-substituted HIV-1 Tat (47-57), arginine-substituted HIV-1 Tat (47-57), Drosophila antennapedia (43-58), a viral RNA-binding peptide containing 7 or more amino acids, a DNA-binding peptide containing 7 or more arginines, a polyarginine polypeptide having 6 to 8 arginines, and a polylysine polypeptide having 7 to 11 lysines.

Most cell-penetrating peptides are known to have high in-vitro penetration efficiency for various cell lines, and it can be predicted that binding of cargo proteins thereto will increase cell permeability. However, it has been generally confirmed that the cell-penetrating peptides have much lower penetration efficiency for primary cells, which has greatly limited the clinical application of the cell-penetrating peptides in humans (Simon, M. J., Gao, S., Kang, W. H., Banta, S. & Morrison, B., 3rd. TAT-mediated intracellular protein delivery to primary brain cells is dependent on glycosaminoglycan expression. Biotechnology and Bioengineering 104, 10-19, doi: 10.1002/bit.22377 (2009)). Meanwhile, according to the present disclosure, it was confirmed that the effect of clinical application in humans, which was previously unpredictable, was increased significantly by conjugating a cell-penetrating peptide to the peptide represented by the amino acid sequence of SEQ ID NO: 4. In particular, it was confirmed that the activity of PPI inhibition between IP₃R and GRP75 and autophagy increasing activity in the human body were increased, and thus the efficacy for a disease related to autophagy disorder was increased significantly, when the cell-penetrating peptide was a peptide represented by the amino acid sequence of SEQ ID NO: 5 (Hph-1).

In a specific exemplary embodiment, the fusion peptide may be represented by any amino acid sequence selected from SEQ ID NO: 24 and SEQ ID NOS: 30 to 44.

In a specific exemplary embodiment, the fusion peptide may have one or more characteristics selected from 1) to 4):
1) inhibition of calcium transport from the endoplasmic reticulum to mitochondria;
2) increase in lysosomal activity;
3) increase in autophagic activity; and
4) increased distance between the endoplasmic reticulum and mitochondria.

Another aspect of the present disclosure relates to a nucleic acid molecule encoding the fusion peptide.

In this specification, the term “nucleic acid molecule” encompasses DNA (gDNA and cDNA) and RNA molecules, and nucleotides, which are basic structural units in nucleic acid molecules, include not only natural nucleotides, but also analogues with modified sugar or base moieties (Scheit, Nucleotide Analogs, John Wiley, New York (1980); Uhlman and Peyman, Chemical Reviews, 90:543-584 (1990)). The sequence of the nucleic acid molecule encoding the fusion protein of the present disclosure may be modified. The modification includes addition, deletion, or non-conservative or conservative substitution of nucleotides.

The nucleic acid molecule of the present disclosure is also interpreted to include a nucleotide sequence showing substantial identity to the above-mentioned nucleotide sequence. The substantial identity means a nucleotide sequence that exhibits at least 80% homology, specifically at least 90% homology, more specifically at least 95% homology, when the nucleotide sequence of the present disclosure is aligned with other sequence to the greatest extent possible and the aligned sequences are analyzed using an algorithm commonly used in the art.

According to a specific exemplary embodiment of the present disclosure, the nucleic acid molecule may contain a nucleotide sequence encoding the fusion peptide.

According to a specific exemplary embodiment of the present disclosure, the nucleic acid molecule may encode any amino acid sequence selected from SEQ ID NO: 24 and SEQ ID NOS: 30 to 44.

Another aspect of the present disclosure relates to an expression vector containing the nucleic acid molecule.

The term “vector” as used herein means a vehicle capable of inserting a polynucleotide sequence for introduction into a cell that can replicate the nucleic acid molecule sequence. The polynucleotide sequence may be exogenous or heterologous. The vector may be, for example, a plasmid, a cosmid vector, or a viral vector (e.g., retrovirus, adenovirus, adeno-associated virus, etc.), although not being limited thereto. One skilled in the art can construct the vector according to standard recombinant techniques (Maniatis, et al., Molecular Cloning, A Laboratory Manual, Cold Spring Harbor Press, Cold Spring Harbor, NY, 1988; and Ausubel et al., In: Current Protocols in Molecular Biology, John, Wiley & Sons, Inc, NY, 1994, etc.).

As used herein, the term “expression construct” means a vector containing a nucleotide sequence encoding at least a portion of a gene product to be transcribed. In some cases, the RNA molecule is then translated into a protein, a polypeptide or a peptide. The expression vector can contain a variety of regulatory sequences. In addition to a regulatory sequence that regulates transcription and translation, the vector and the expression vector may also contain a nucleic acid sequence that provides other functions.

Another aspect of the present disclosure relates to a host cell containing the vector or expression construct described above or transformed with the vector or expression construct.

In this specification, the term “cell” includes eukaryotic and prokaryotic cells, and refers to any transformable cell capable of replicating the vector described above or expressing a gene encoded by the vector. The cell can be transfected, transduced or transformed by the vector, which refers to a process by which an exogenous polynucleotide (nucleic acid molecule) is delivered or introduced into the host cell. In this specification, the term “transformation” is used to include transfection and transduction.

The (host) cell of the present disclosure includes, specifically an insect cell or a mammalian cell, more specifically an insect cell such as Sf9 cell, or a mammalian cell such as HEK293 cell, HeLa cell, ARPE-19 cell, RPE-1 cell, HepG2 cell, Hep3B cell, Huh-7 cell, C8D1a cell, Neuro2A cell, CHO cell, MES13 cell, BHK-21 cell, COS7 cell, COP5 cell, A549 cell, MCF-7 cell, HC70 cell, HCC1428 cell, BT-549 cell, PC3 cell, LNCaP cell, Capan-1 cell, Panc-1 cell, MIA PaCa-2 cell, SW480 cell, HCT166 cell, LoVo cell, A172 cell, MKN-45 cell, MKN-74 cell, Kato-III cell, NCI-N87 cell, HT-144 cell, SK-MEL-2 cell, SH-SY5Y cell, C6 cell, HT-22 cell, PC-12 cell, NIH3T3 cell, etc., although not being limited thereto.

Specifically, the host cell of the present disclosure is an isolated host cell.

Another aspect of the present disclosure relates to a pharmaceutical composition for preventing or treating a disease related to autophagy disorder, which contains the fusion peptide, the nucleic acid molecule, the expression vector or the host cell as an active ingredient.

Since the “fusion peptide” of the present disclosure has already been described above, its description is omitted to avoid excessive redundancy.

As used herein, the term “disease related to autophagy disorder” refers to any disease caused by the generation of abnormal cells or proteins that are not removed by autophagy. For example, it includes a disease in which there is a defect in the maturation of autophagosomes required for autophagy.

As used herein, the term “abnormal cell” means a cell that is significantly distinguished from a normal cell either morphologically or functionally. The abnormal cell includes all of cells that are malnourished, cells that are genetically altered and have impaired morphology and function, and cells that are physically damaged, etc.

As used herein, the term “abnormal protein” means a protein that is significantly distinguished from a normal protein either morphologically or functionally. The abnormal protein includes all of aggregate-prone mutant proteins whose tertiary structure has been altered by amino acid mutations, etc.

In a specific exemplary embodiment, the disease related to autophagy disorder may be a disease caused by abnormal protein accumulation or a degenerative disease.

In a specific exemplary embodiment, the disease caused by abnormal protein accumulation or the degenerative disease may be any one selected from arteriosclerosis, pulmonary hypertension, Alzheimer's disease, Parkinson's disease, type 2 diabetes, amyotrophic lateral sclerosis, dialysis-related amyloidosis, cystic fibrosis, sickle cell anemia, Huntington's disease, Creutzfeldt-Jakob disease, Lewy body dementia, inclusion body myositis, cerebral amyloid angiopathy, traumatic brain injury, frontotemporal dementia, progressive supranuclear palsy, corticobasal degeneration, Pick's disease, and argyrophilic grain disease, induced by reduced intracellular autophagy.

In a specific exemplary embodiment, the arteriosclerosis may be atherosclerosis.

In a specific exemplary embodiment, the composition may reduce intracellular lipids by inducing autophagy.

The fusion peptide of the present disclosure has strong cell membrane permeability and does not induce an immune response to CPP when applied to the human body. In addition, it was confirmed through specific experiments that the fusion peptide according to the present disclosure does not exhibit hemolytic activity and thus has no cytotoxicity issue. In addition, the fusion peptide of the present disclosure induces intracellular autophagy by regulating the activity of MAM by inhibiting PPI (protein-protein interaction) between IP₃R and GRP75.

According to a specific example of the present disclosure, when the fusion peptide of the present disclosure was administered to an atherosclerosis-induced animal model (ApoE−/− mice fed with a high-cholesterol diet), it was confirmed that atherosclerotic plaques in the aorta were significantly reduced and the lesion area of the aorta was significantly reduced as compared to a control group. This suggests that the fusion peptide of the present disclosure is effective in ameliorating or alleviating a disease related to autophagy disorder, particularly atherosclerosis.

In the present disclosure, the term “prevention” means any action that suppresses or delays a disease related to autophagy disorder.

The term “treatment” of the present disclosure, unless stated otherwise, means reversing, alleviating, or inhibiting or preventing the progression of the symptoms of a disease related to autophagy disorder.

The pharmaceutical composition of the present disclosure may be prepared using a pharmaceutically adequate and physiologically acceptable adjuvant in addition to the active ingredient. The adjuvant may include an excipient, a disintegrant, a sweetener, a binder, a coating agent, an extender, a lubricant, a glidant, a flavoring agent, etc.

The pharmaceutical composition may be specifically formulated as a pharmaceutical composition containing one or more pharmaceutically acceptable carrier in addition to the active ingredient described above for administration.

The pharmaceutical composition may be in the form of a granule, a powder, a tablet, a coated tablet, a capsule, a suppository, a liquid, a syrup, a juice, a suspension, an emulsion, a medicinal drop, an injectable liquid, etc. For example, for formulation into a tablet or a capsule, the active ingredient may be combined with an oral, nontoxic, pharmaceutically acceptable inert carrier such as ethanol, glycerol, water, etc. Furthermore, if desired or necessary, a suitable binder, a lubricant, a disintegrant and a colorant may also be included as a mixture. The binder includes natural sugars such as starch, gelatin, glucose, or beta-lactose, corn sweetener, natural and synthetic gums such as acacia, tragacanth or sodium oleate, sodium stearate, magnesium stearate, sodium benzoate, sodium acetate, sodium chloride, etc., although not being limited thereto. The disintegrant includes starch, methyl cellulose, agar, bentonite, xanthan gum, etc., although not being limited thereto.

A composition formulated as a liquid solution may contain, as a pharmaceutically acceptable carrier which is sterile and biocompatible, saline, sterile water, Ringer's solution, buffered saline, albumin injection solution, dextrose solution, maltodextrin solution, glycerol, ethanol, and mixtures of one or more of these ingredients. Other common additives such as an antioxidant, a buffer, a bacteriostatic agent, etc. may be added, if necessary. In addition, the composition may also be formulated into an injectable formulation such as an aqueous solution, a suspension, an emulsion, etc., a pill, a capsule, a granule or a tablet by further adding a diluent, a dispersant, a surfactant, a binder and a lubricant.

Furthermore, the composition may be formulated, depending on the relevant disease or the ingredients, using suitable methods in the art as disclosed in Remington's Pharmaceutical Science (Mack Publishing Company, Easton PA).

The pharmaceutical composition of the present disclosure can be administered orally or parenterally, and in the case of parenteral administration, it can be administered by intravenous injection, subcutaneous injection, intramuscular injection, intraperitoneal injection, transdermal administration, etc.

The pharmaceutical composition of the present disclosure can provide a desirable effect of preventing, alleviating or treating a disease related to autophagy disorder when it contains an effective amount of the fusion peptide, the nucleic acid molecule, the expression vector or the host cell. As used herein, the “effective amount” refers to an amount that elicits a response greater than that of a negative control group, specifically an amount sufficient to alleviate or treat a disease related to autophagy disorder. The fusion peptide may be contained at a concentration of 0.5 to 500 μM, specifically 1 to 400 μM, more specifically 10 to 300 μM, more specifically 50 to 200 μM, based on the total content of the pharmaceutical composition. If the content of the fusion peptide is below the lower limit, although cell viability may be excellent, the effect of alleviating or treating a disease related to autophagy disorder may not be satisfactory. Conversely, if the concentration exceeds the upper limit, the effect of alleviating or treating a disease related to autophagy disorder may not be so high as desired, or toxicity may occur.

The total effective amount of the pharmaceutical composition of the present disclosure can be administered to a patient as a single dose, or can be administered by a fractionated treatment protocol in which multiple doses are administered over a long period of time. The pharmaceutical composition of the present disclosure may contain different contents of the active ingredient depending on the severity of the disease.

The appropriate dosage of the pharmaceutical composition varies depending on factors such as formulation method, administration method, the patient's age, body weight, sex, pathological condition and diet, administration time, administration route, excretion rate, and response sensitivity, and a generally skilled physician can easily determine and prescribe a dosage effective for the desired treatment or prevention. According to a specific exemplary embodiment, the preferred daily dosage of the pharmaceutical composition may be 0.5 to 500 mg/kg, specifically 1 to 250 mg/kg, more specifically 1 to 100 mg/kg, more specifically 5 to 50 mg/kg.

Another aspect of the present disclosure relates to a food composition for preventing or alleviating a disease related to autophagy disorder, which contains the fusion peptide, the nucleic acid molecule, the expression vector or the host cell as an active ingredient.

Since the “fusion peptide”, “autophagy”, and “disease related to autophagy disorder” of the present disclosure have already been described above, their description will be omitted to avoid excessive redundancy.

The food composition according to the present disclosure can be formulated as a powder, a granule, a tablet, a capsule, etc. using a sitologically adequate and physiologically acceptable adjuvant for use as a functional food. The adjuvant may include an excipient, a disintegrant, a sweetener, a binder, a coating agent, an extender, a lubricant, a glidant, a flavoring agent, etc.

Furthermore, the food composition according to the present disclosure may be, for example, a beverage, an alcoholic beverage, confectionery, a diet bar, a dairy product, meat, chocolate, pizza, ramen, other noodles, a chewing gum, ice cream, etc.

The food composition of the present disclosure may contain not only CTS as an active ingredient, but also ingredients commonly added during food preparation including, for example, a protein, a carbohydrate, a fat, a nutrient, a seasoning, and a flavoring agent. Examples of the carbohydrate include: common sugars such as monosaccharides, e.g., glucose, fructose, etc.; disaccharides, e.g., maltose, sucrose, etc.; oligosaccharides; polysaccharides, e.g., dextrin, cyclodextrin, etc., and sugar alcohols such as xylitol, sorbitol, erythritol, etc. As the flavorant, natural flavorants [thaumatin or stevia extract (e.g., rebaudioside A, glycyrrhizin, etc.)] and synthetic flavorants (saccharin, aspartame, etc.) can be used. For example, when the food composition of the present disclosure is prepared into a drink or a beverage, citric acid, high-fructose corn syrup, sugar, glucose, acetic acid, malic acid, fruit juice, various plant extracts, etc. may be additionally included in addition to CTS of the present disclosure.

Another aspect of the present disclosure relates to a composition for activating autophagy in vitro, which contains the fusion peptide, the nucleic acid molecule, the expression vector or the host cell as an active ingredient.

Since the “fusion peptide” of the present disclosure has already been described above, its description is omitted to avoid excessive redundancy.

In a specific exemplary embodiment, the composition of the present disclosure can enhance autophagic activity in a cell in vitro.

The cell may be a cancer cell, a vascular endothelial cell (HUVEC), or a macrophage.

Another aspect of the present disclosure relates to a composition for inhibiting the interaction of the endoplasmic reticulum and mitochondria in vitro, which contains the fusion peptide, the nucleic acid molecule, the expression vector or the host cell as an active ingredient.

Since the “fusion peptide” of the present disclosure has already been described above, its description is omitted to avoid excessive redundancy.

In a specific exemplary embodiment, the composition of the present disclosure reduces contact between the endoplasmic reticulum and mitochondria in cells in vitro, increases the distance between the endoplasmic reticulum and mitochondria, and thereby inhibits interactions between these organelles.

The cell may be a cancer cell, a vascular endothelial cell (HUVEC), or a macrophage.

Hereafter, the present disclosure will be described in more detail through examples. These examples are intended solely to explain the present disclosure in more detail, and it will be obvious to those having common knowledge in the art that the scope of the present disclosure is not limited by the examples.

### EXAMPLES

### <Experimental Methods>

### Design and Synthesis of Fusion Peptides

Macromolecules such as proteins and peptides have difficulty in penetrating into cells. Cell-penetrating peptides (CPPs) were introduced to solve this problem. CPPs are peptides consisting of less than about 30 amino acids, which do not destroy cell membranes, do not exhibit cytotoxicity, and can pass through cell membranes to deliver hydrophilic macromolecules into the cytoplasm and nucleoplasm.

Based on the analysis of structural information and bioinformatics of complexes, four different types of fusion peptides were designed by linking a CPP sequence that increases cell membrane permeability for a part of the IP₃R protein motif that is expected to regulate protein-protein interaction (PPI) with GRP75, or for a part of the GRP75 protein motif that is expected to regulate PPI with IP₃R.

Specifically, fusion peptides 1 to 4 (SEQ ID NOS: 21 to 24) were prepared by linking a part of the IP₃R protein motif predicted to regulate PPI with GRP75, or a part of the GRP75 protein motif predicted to regulate PPI with IP₃R (Table 1, SEQ ID NOS: 1 to 4), and a cell-penetrating peptide (Table 2, SEQ ID NO: 5) by peptide bonds (Table 3). The fusion peptides 1 to 4 were tested through experiments.

In addition, the entire amino acid sequence of the IP₃R protein (SEQ ID NO: 25), the predicted PPI sequence in the IP₃R protein (SEQ ID NO: 26), the entire amino acid sequence of the GRP75 protein (SEQ ID NO: 27), and the predicted PPI sequences in the GRP75 protein (SEQ ID NO: 28 and SEQ ID NO: 29) are shown in Table 4.

TABLE 1

| SEQ ID NO | | Sequence |
|---|---|---|
| SEQ ID NO: 1 | Peptide 1 | FSTAADGQTQ |
| SEQ ID NO: 2 | Peptide 2 | GGG-FSTAADGQTQ |
| SEQ ID NO: 3 | Peptide 3 | DTETKME |
| SEQ ID NO: 4 | Peptide 4 | RSIYYLGIGPT |

TABLE 2

| SEQ ID NO | | CPP sequence |
|---|---|---|
| SEQ ID NO: 5 | Hph-1 | YARVRRRGPRR |
| SEQ ID NO: 6 | Transportan | GWTLNSAGYLLGKINLKALAALAKKIL |
| SEQ ID NO: 7 | Pep-1 | KETWWETWWTEWSQPKKKRKV |
| SEQ ID NO: 8 | pVEC | LLIILRRRIRKQAHAH |
| SEQ ID NO: 9 | M918 | MVTVLFRRLRIRRACGPPRVRV |
| SEQ ID NO: 10 | TP10 | AGYLLGKINLKALAALAKKIL |
| SEQ ID NO: 11 | VP22 | DAATATRGRSAASRPTERPRAPARSASRPRRPVE |
| SEQ ID NO: 12 | Buforin 2 | TRSSRAGLQFPVGRVHRLLR |
| SEQ ID NO: 13 | KALA | WEAKLAKALAKALAKHLAKALAKALKACEA |
| SEQ ID NO: 14 | CL22 | KKKKKKGGFLGFWRGENGRKTRSAYERMCNILKGK |
| SEQ ID NO: 15 | Crotamine | YKQCHKKGGHCFPKEKICLPPSSDFGKMDCRWRW |
| | | KCCKKGSG |
| SEQ ID NO: 16 | IRGD | CRGDKGPDC |
| SEQ ID NO: 17 | dNP2 | KIKKVKKKGRKGSKIKKVKKKGRK |
| SEQ ID NO: 18 | mdNP2 | KIKKRKKKGRKGSKIKKVKKKGRK |
| SEQ ID NO: 19 | mmdNP2 | KIKKRKKKGRKGSKIKKRKKKGRK |
| SEQ ID NO: 20 | 9R | RRRRRRRRR |

TABLE 3

| | | CPP (Hph-1, SEQ ID NO: 5) - | Molecular |
|---|---|---|---|
| SEQ ID NO | | sequence in Table 1 | weight (g/mol) |
| SEQ ID NO: 21 | Fusion Peptide 1 | YARVRRRGPRR-FSTAADGQTQ | 2450 |
| SEQ ID NO: 22 | Fusion Peptide 2 | YARVRRRGPRR-GGG- | 2620 |
| | | FSTAADGQTQ | |
| SEQ ID NO: 23 | Fusion Peptide 3 | YARVRRRGPRR-DTETKME | 2278 |
| SEQ ID NO: 24 | Fusion Peptide 4 | YARVRRRGPRR-RSIYYLGIGPT | 2664 |

TABLE 4

| SEQ ID NO | Protein | Domain | Sequence |
|---|---|---|---|
| SEQ ID NO: 25 | IP₃R | Transmembrane | MPLIYWFSRRMTLWGSISFNLAVFINIIIA |
| | | domain | FFYPYMEGASTGVLDSPLISLLFWILICFS |
| | | (Amino acids | IAALFTKRYSIRPLIVALIL RSIYYLGIGPT |
| | | 2191-2537) | LNILGALNLTNKIVFVVSFVGNRGTFIRG |
| | | | YKAMVMDMEFLYHVGYILTSVLGLFAHE |
| | | | LFYSILLFDLIYREETLFNVIKSVTRNGRSI |
| | | | LLTALLALILVYLFSIVGFLFLKDDFILEVD |
| | | | RLPNNHSTASPLGMPHGAAAFVDTCSG |
| | | | DKMDCVSGLSVPEVLEEDRELDSTERA |
| | | | CDTLLMCIVTVMNHGLRNGGGVGDILRK |
| | | | PSKDESLFPARVVYDLLFFFIVIIIVLNLIF |
| | | | GVIIDTFADLRSEKQKKEEILKTT |
| SEQ ID NO: 26 | IP₃R | Protein-protein | RSIYYLGIGPT |
| | | interaction (PPI) | |
| | | of IP₃R | |
| | | Predicted | |
| | | sequence | |
| SEQ ID NO: 27 | GRP75 | Substrate- | DVTPLSLGIETLGGVFTKLINRNTTIPTKK |
| | | binding domain | SQV FSTAADGQTQ VEIKVC |
| | | (Amino acids | QGEREMAGDNKLLGQFTLIGIPPAPRGV |
| | | 439-597) | PQIEVTFDIDANGIVHVSAKD |
| | | | KGTGREQQIVIQSSGGLSKDDIENMVKN |
| | | | AEKYAEEDRRKKERVEAVNM AEGIIH |
| | | | DTETKME |
| SEQ ID NO: 28 | GRP75 | Predicted | FSTAADGQTQ |
| | | sequence of PPI | |
| | | in GRP75 (1) | |
| SEQ ID NO: 29 | GRP75 | Predicted | DTETKME |
| | | sequence of PPI | |
| | | in GRP75 (2) | |

### MTT Assay

Human umbilical vein endothelial cells (HUVECs) were cultured in EBM-2 medium supplemented with FBS (2%, v/v) and antibiotics. After seeding the cells in a 96-well plate at 3,000 cells/well and culturing the same, they were treated with the prepared fusion peptides on the next day at concentrations of 0, 100, 200, and 300 μM. After 0, 24, 48, and 72 hours, MTT solution (2 mg/mL) was treated at a concentration of 0.4 mg/mL. After culturing for 3 hours, the medium was removed and the cells were treated with DMSO. Then, fluorescence was measured at 540 nm.

### Measurement of Ca²⁺ Movement in ER-Mitochondria

Hela cells were seeded in an 8-well glass slide at 1.5×10⁴ cells/well, and cultured for 24 hours. Afterwards, the cells were treated with each of the fusion peptides of the present disclosure at a concentration of 20 μM for 1 hour, and then washed twice with KRH (Krebs-Ringer-HEPES) buffer. Afterwards, Rhod-2 dye diluted in KRH buffer was added. After incubation at room temperature for 30 minutes, the cells were washed three times with Ca²⁺-free KRH buffer. Afterwards, the cells were imaged at 5-second intervals for 3 minutes using a confocal microscope LSM880 set to time series. The ratio of fluorescence area to cell area per each cell was quantified using the Image J program, and a graph was drawn by normalization by dividing all values (F) by the initial value (FO).

### PLA (Proximity Ligation Assay)

HeLa cells were cultured in a chamber-type cell culture slide and treated with each of the fusion peptides of the present disclosure at a concentration of 20 μM for 1 hour. Afterwards, PLA assay was performed using a Duolink in-situ red kit (Sigma-Aldrich, St. Louis, MO, DUO92101) according to the manufacturer's instructions. Specifically, the cells were fixed with 4% PFA for 10 minutes and permeabilized at room temperature with 0.2% Triton X-100 for 20 minutes. Afterwards, the cells were blocked for 1 hour using a blocking solution and then incubated overnight at 4° C. with primary antibodies targeting IP₃R in the endoplasmic reticulum (1:100 dilution) and VDAC1 in the mitochondria (1:200 dilution), respectively, derived from different species. Afterwards, the cells were incubated with PLA probes (anti-rabbit PLUS and anti-mouse MINUS) at 37° C. for 1 hour. The cells were incubated with ligase for 30 minutes and then with DNA polymerase at 37° C. for 100 minutes. Afterwards, the sample was mounted using a mounting medium containing DAPI. Then, PLA signals were observed using a Zeiss LSM 980 confocal microscope, and the number of PLA dots was counted and quantified.

### Immunoblot Assay

The cell culture was obtained and the expression of proteins was confirmed by immunoblot assay.

Specifically, the cells were lysed using SDS lysis buffer (50 mM Tris-HCl [pH 6.8] containing 10% glycerol, 2% SDS, 10 mM dithiothreitol, and 0.005% bromophenol blue) to obtain a cell lysate. After centrifugation of the cell lysate, the proteins in the supernatant were quantified using the Bradford method (Bio-Rad Laboratories Inc., Hercules, CA, USA). The quantified proteins were separated by 11.5% SDS-PAGE (resolving buffer 1.5 M Tris-Cl, pH 8.8, stacking buffer 0.5 M Tris-CI, pH 6.8) and transferred to a PVDF (polyvinylidene difluoride) membrane (BioRad, USA). The membrane onto which the proteins were transferred was incubated overnight at 4° C. with primary antibodies [anti-SQSTM1/SQSTM1 (5114s, CST), anti-LC3B (2775s, CST), anti-myc, anti-TFEB, anti-β-actin (ab6276, Abcam), and anti-β-tubulin (Abcam)] diluted with 3% (w/v) skim milk or 5% (w/v) BSA. Afterwards, the membrane was washed with TBST buffer (10 mM Tris-HCl, 150 mM NaCl, 0.05% Tween 20, pH 7.4), and incubated with appropriate secondary antibodies (1:3000 v/v, GE Healthcare) at 25° C. for 1 hour. Afterwards, the proteins were detected using an enhanced chemiluminescence substrate (Bio-Rad) according to the manufacturer's instructions, and images were captured using Image Lab (Bio-Rad, Hercules, CA, USA). ACTB was used as an internal control group.

### ICC (Immunocytochemistry) Assay

Hela cells were seeded onto a glass coverslip at 1.5×10⁴ cells/well and then cultured. The next day, the cells were treated with the fusion peptides of the present disclosure at concentrations of 20 μM and 50 μM for 0, 0.5, 1, 3, 6, and 12 hours, respectively. After washing with PBS, the cells were fixed by treating with 4% paraformaldehyde for 10 minutes at room temperature. After washing three times with PBS, the cells were treated with 0.2% Triton X-100 for 10 minutes at room temperature for permeabilization. After washing with PBS, the cells were blocked with 1% BSA for 30 minutes at room temperature. The cells were treated with TFEB antibodies diluted 1:50 in 1% BSA and incubated overnight at 4° C. The next day, the cells were washed three times for 5 minutes each with PBS, and treated with fluorochrome-labeled secondary antibodies diluted in 1% BSA (1:500) for 1 hour at room temperature. After washing three times with PBS for 3 minutes each, the cells were mounted using a mounting solution containing DAPI. Colocalization of DAPI and TFEB used for staining the nuclei was quantified using the Image J software.

### Fluorescence Staining

HUVEC cells were seeded in an 8-well glass cell slide at 1.5×10⁴ cells/well and cultured for 24 hours. Then, they were treated with the fusion peptide of the present disclosure at a concentration of 100 μM for 12 or 24 hours. After removing the medium, the cells were washed with PBS and treated with 200 nM LysoTracker Deep Red for 20 minutes. The cells were fixed with 4% PFA for 10 minutes and washed three times with PBS. The cells were mounted by treating with a mounting solution containing DAPI. Fluorescence images were captured at 400× magnification using an LSM 980 confocal microscope. Fluorescence intensity per cell was quantified using the Image J software.

### Animal Experiment

Animal experiment was performed in accordance with the guidelines approved by the Institutional Animal Care and Use Committee of Yonsei University (IACUC-A-202404-1847-02). 6-week-old male ApoE knockout B6.KOR/StmSlc-Apoeshl mice were housed in a specific pathogen-free system with a 12-hour light-dark cycle with free access to food and water, and randomly divided into three groups (control, positive control, and test groups). Then, the sample was injected intraperitoneally to the control group (vehicle), the positive control group (CTS (cryptotanshinone), 20 mg/kg), and the test group (peptide, 50 mg/kg) once every two days for 8 weeks while feeding the mice with a high-cholesterol diet until the end of the experiment (for 8 weeks).

After sacrificing the mice, PBS perfusion was performed through the left ventricle, and the heart, artery, and blood were obtained. The artery was cut along the bifurcation of the iliac artery, spread out, fixed with pins, and fixed in 10% formaldehyde for 16 hours. After staining with Oil-Red-O for 16 hours, the samples were washed with PBS and images were obtained using a microscope (NS-6T, China).

### <Experimental Results>

### Test Example 1: Production of Fusion Peptide Inhibiting Interaction Between IP₃R and GRP75

Unlike orthosteric modulators that act on the active site of a protein, allosteric modulators that act on sites other than the active site are attracting attentions as a recent drug development strategy since they exhibit high target selectivity, few side effects, and low toxicity. Therefore, the domains of IP₃R and GRP75 were analyzed to discover peptides targeting allosteric sites as a strategy to modulate protein-protein interactions (PPIs).

Based on the analysis of structural information and bioinformatics of complexes, four different types of fusion peptides were designed and produced by linking the Hph-1 sequence, which is a CPP sequence that increases cell membrane permeability, to a part of the IP₃R sequence or to a part of the GRP75 sequence, which are expected to regulate the interaction between IP₃R and GRP75, and then their activities were evaluated (Table 1). The human transcriptional factor Hph-1 contains a cell permeability domain (YARVRRRGPRR) containing positively charged arginine and lysine amino acid residues, and thus facilitates contact with the negatively charged cell membrane. Meanwhile, CPP derived from proteins derived from viruses (Tat, GRKKRRQRRRPPQ) or fruit flies (Antp, RQIKIWFQNRRMKWKK) has a possibility of inducing an immune response when applied to humans, which may reduce the efficacy of drugs.

### Test Example 2: Discovery of Novel Fusion Peptides with MAM Regulatory Activity

It was investigated whether the four types of fusion peptides produced in the Test Example 1 inhibit the IP₃R-GRP75 interaction by binding to the allosteric site of IP₃R or GRP75, thereby reducing the proximity of the endoplasmic reticulum and mitochondria.

To this end, MAM regulatory activity was verified using the SPLICS (ER-mitochondria split-GFP) system, which allows observation of the proximity between intracellular organelles through fluorescence emission. The SPLICS system is a system that can efficiently image the proximity of intracellular organelles based on the principle that split-GFP fluorescence is emitted when two organelles are within approximately 10 to 40 nm of each other. As the two organelles approach, complementation of GFP occurs, resulting in fluorescence at the junction. It is to be noted that increased contact between ER and mitochondria may lead to cellular stress and dysfunction.

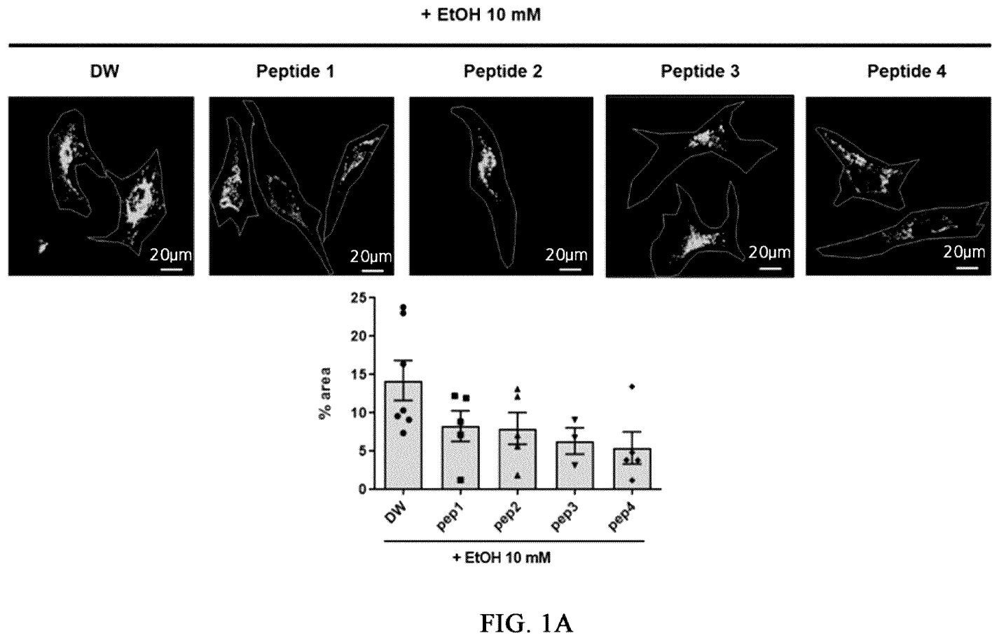

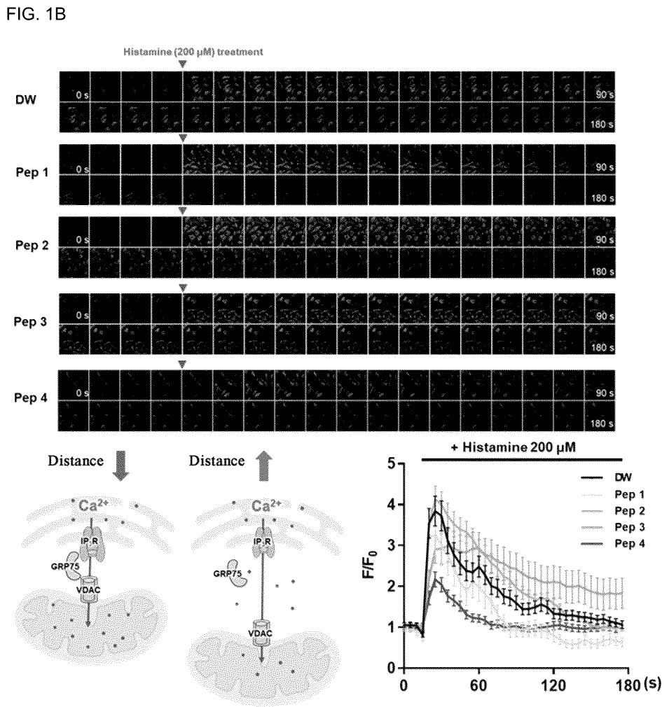

First, to observe the change in the distance between the ER and mitochondria due to treatment with the fusion peptides, Hela cells were transfected with the SPLICS vector. Afterwards, the HeLa cells were treated with the fusion peptides and the area (%) of GFP fluorescence relative to the total area per cell was measured. Looking at FIG. 1A, it can be confirmed that all the groups treated with the fusion peptides of the present disclosure (pep1, pep2, pep3, pep4) showed significant decrease in fluorescence signals as compared to the control group (DW). That is to say, it was observed from the decrease in green fluorescence in the groups treated with the fusion peptides of the present disclosure that the two organelles were moving apart from each other.

Additionally, for further verification, mitochondrial calcium level was determined by fluorescence. If calcium release from the endoplasmic reticulum is induced by treating with histamine, which is an agonist of IP₃R, calcium influx into the mitochondria increases when the distance between the endoplasmic reticulum and the mitochondria is close as compared to when the distance is far. Rhod2 dye, which is a calcium indicator, binds to calcium inside mitochondria and fluoresces. So, the amount of calcium inside mitochondria can be determined by the intensity of fluorescence.

Looking at FIG. 1B, it can be confirmed that the group treated with the fusion peptide 4 of the present disclosure (pep4) exhibits a significantly reduced mitochondrial calcium level as compared to the control group (DW) and the groups treated with the fusion peptides 1 to 3 (pep1, pep2, pep3).

This result suggests that the fusion peptide 4 reduces contact between the ER and mitochondria, thereby attenuating calcium transport from the ER to mitochondria.

That is, the fusion peptide 4 of the present disclosure binds to the allosteric site of IP₃R and inhibits PPI between IP₃R and GRP75.

### Test Example 3: Validation of MAM Regulatory Activity of Fusion Peptide 4

As another method to confirm the change in MAM after treatment with the fusion peptide, proximity ligation assay (PLA), which can visualize the proximity between two proteins, was performed. PLA is a technique based on the fact that fluorescence is emitted when two proteins come within a distance of approximately 40 nm, making it an effective experimental method for observing dynamic protein-protein interactions.

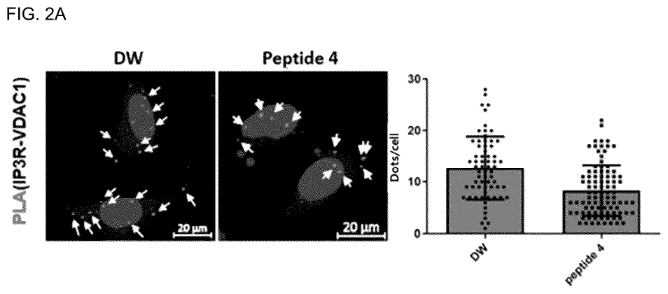

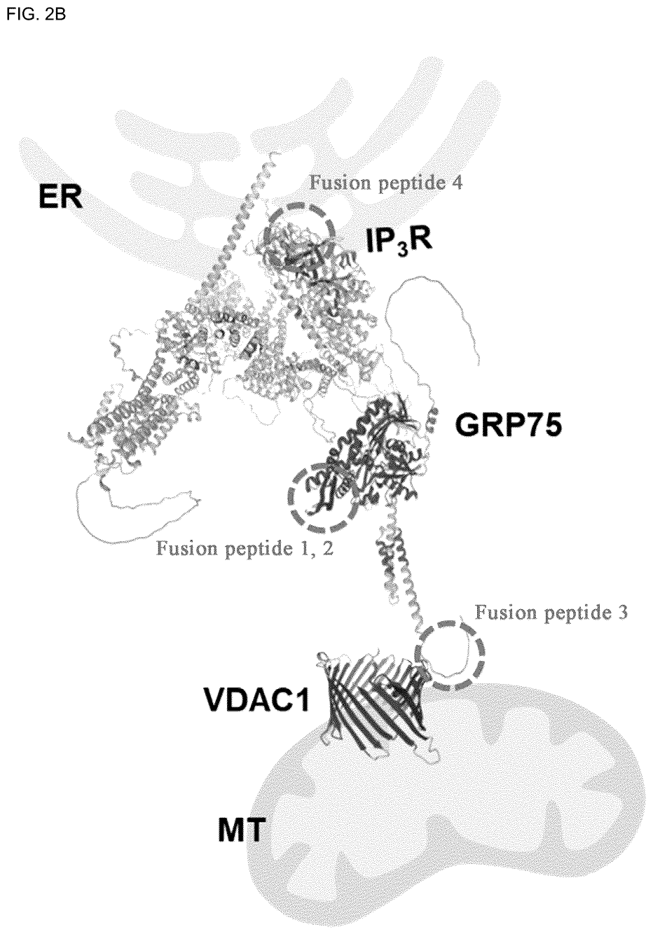

Specifically, Hela cells were treated with the fusion peptide 4 (20 μM) for 1 hour, and then PLA was performed using organelle markers. As a result, the number of PLA dots decreased significantly in the group treated with the fusion peptide 4 as compared to the control group, confirming that the fusion peptide 4 increases the distance between mitochondria and the endoplasmic reticulum (FIG. 2A). Through the above verification experiments, among the several fusion peptide candidates, the fusion peptide 4 was selected as a peptide exhibiting MAM regulatory activity, i.e., the activity of inhibiting the interaction between the endoplasmic reticulum and mitochondria or the activity of increasing the distance between the endoplasmic reticulum and mitochondria.

Meanwhile, structural analysis (in silico docking analysis) was performed on the fusion peptide 4. As a result, the fusion peptide 4 of the present disclosure contained the transmembrane domain of IP₃R, and it was predicted that the domain would interact with the substrate-binding domain of GRP75 (Table 2 and FIG. 2B). These results imply that the fusion peptide 4 of the present disclosure inhibits the PPI between IP₃R and GRP75 by interacting with the transmembrane domain of IP₃R.

Therefore, it was verified that fusion peptide 4 can regulate the activity of MAM by inhibiting the PPI (protein-protein interaction) between IP₃R and GRP75.

### Test Example 4: Verification of Intracellular Permeability of Fusion Peptide 4 and Determination of Optimal Concentration

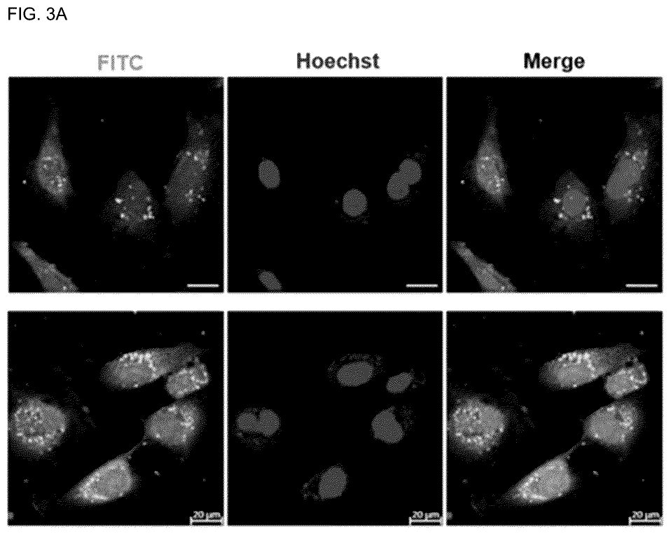

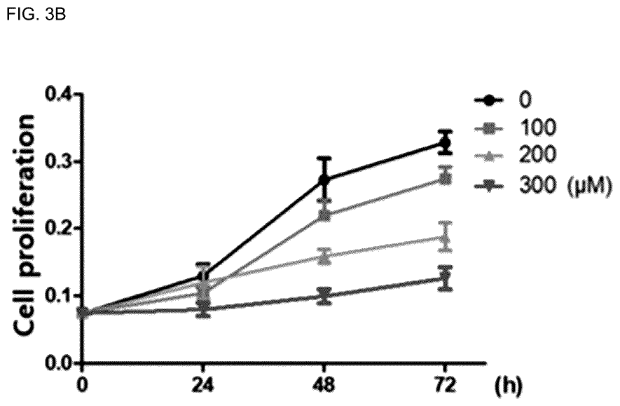

After treating HUVEC cells with the fusion peptide (5 μM, 10 μM) labeled with fluorescein isothiocyanate (FITC) for 3 hours, the cell membrane permeability of the fusion peptide was confirmed using a confocal microscope (FIG. 3A).

Afterwards, 3-[4,5-dimethylthiazol-2-yl]-2,5-diphenyltetrazolium bromide (MTT) assay, which allows observation of cell proliferation by fluorescence, was performed to confirm the optimal concentration of the fusion peptide 4 in the HUVEC cells. Specifically, when the cells were treated with the fusion peptide 4 (0-300 μM) for up to 72 hours, no inhibition of cell proliferation was observed even at concentrations of 100-200 μM (FIG. 3B).

### Test Example 5: Increased TFEB Nuclear Translocation and Lysosomal Activity Following Fusion Peptide 4 Treatment

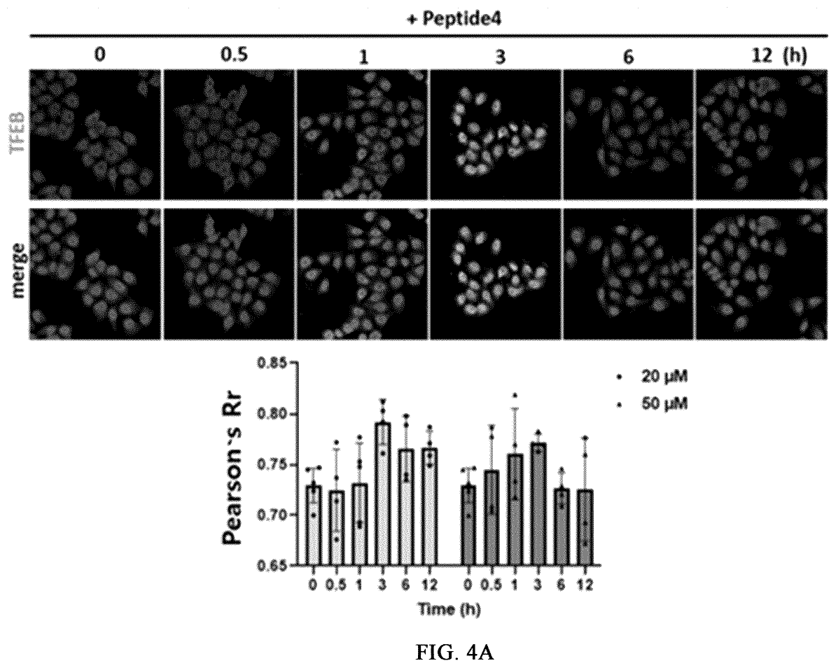

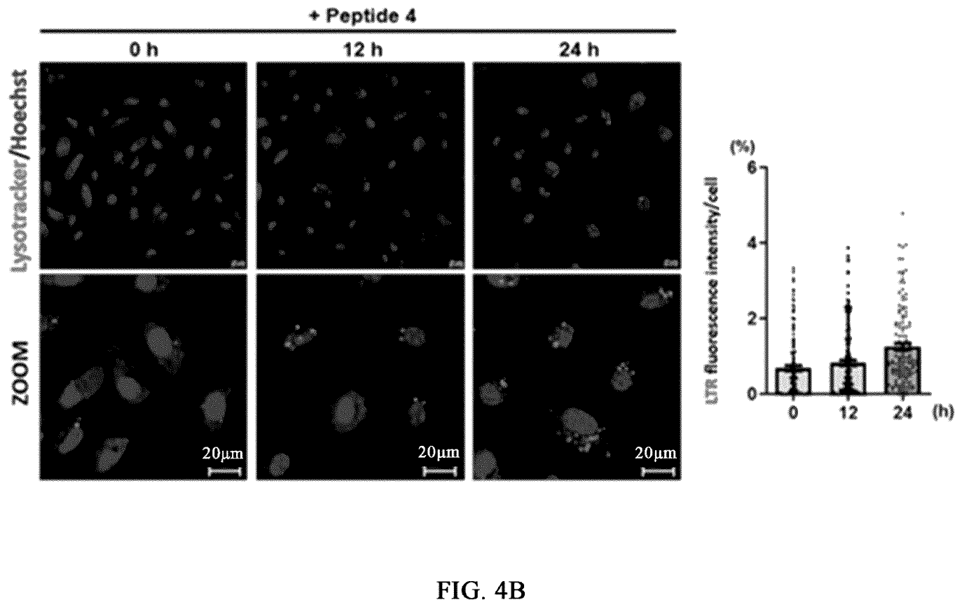

The effect of the change in the interaction of the IP₃R-GRP75-VDAC complex following the treatment with the fusion peptide on autophagic activity was investigated. To observe the change in autophagic activity according to the change in MAM, the nuclear translocation of transcription factor EB (TFEB), which is a transcription factor essential for autophagic and lysosomal activity, was confirmed through immunocytochemistry (ICC) staining. Specifically, after treating Hela cells with the fusion peptide (20 μM, 50 μM) for about 3 hours, the translocation of TFEB to the nucleus was observed (FIG. 4A). This suggests that treatment with the fusion peptide 4 induces intracellular autophagy.

Additionally, to observe lysosomes activated during the autophagy process, lysosomes were stained with Lysotracker, which is a cell-permeable lysosome marker, and then photographed using a confocal microscope. Specifically, when HUVEC cells were treated with the fusion peptide (100 μM) for 12 or 24 hours, lysosomal activity was found to increase (FIG. 4B).

### Test Example 6: Induction of Autophagy Flux by Treatment with Fusion Peptide 4

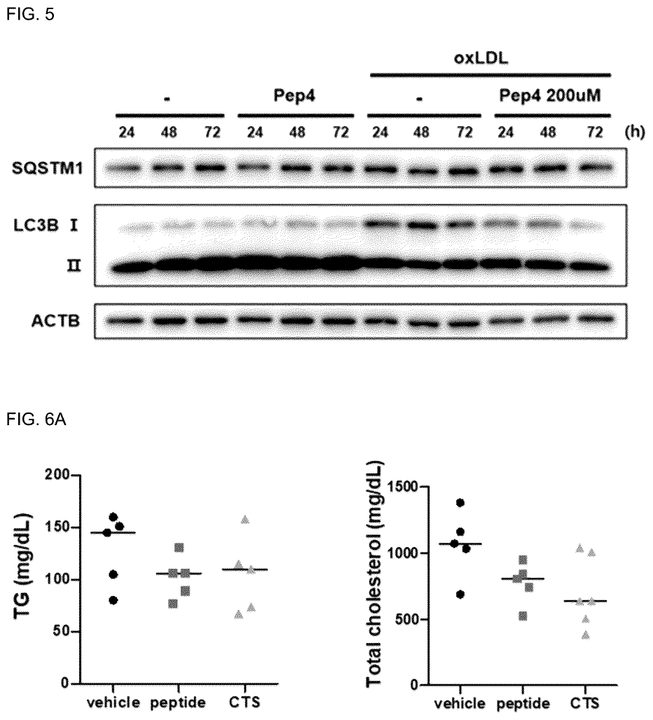

The expression level of LC3 and SQSTM1, which are markers for evaluating autophagic activity, was investigated (FIG. 5). As a result of performing western by treating HUVEC cells with 50 μg/mL of oxidized low-density lipoprotein (oxLDL), the conversion of LC3B I to LC3B II was decreased. When the cells were treated with 50 μg/mL of oxLDL and 200 μM of the fusion peptide 4 together, the conversion of LC3B I to LC3B II was increased. These results indicate that autophagy was inhibited when the cells were treated with oxLDL, and that autophagic activity was restored when the cells were treated with oxLDL and the fusion peptide together. Therefore, it was confirmed that the fusion peptide according to the present disclosure induces autophagy. This result suggests that autophagy can be induced by inhibiting protein-protein interactions involved in MAM formation by treating the fusion peptide of the present disclosure.

### Test Example 7: Atherosclerosis-Alleviating Effect of Fusion Peptide 4

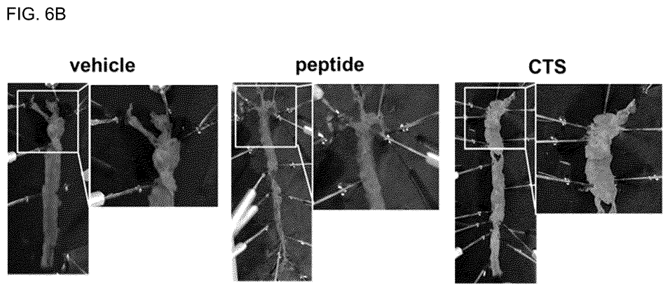

The fusion peptide 4 (50 mg/kg) was injected intraperitoneally every 2 days for 8 weeks into an animal model of atherosclerosis (high-cholesterol diet, ApoE knockout mice). The administration of the fusion peptide 4 did not affect the body weight of the animal model, indicating that it has low toxicity. In addition, the test group administered with the fusion peptide 4 of the present disclosure showed a decrease in serum triglyceride (TG) and total cholesterol levels (FIG. 6A), and a significant decrease in the formation of atherosclerotic plaques in the aorta (FIG. 6B), as compared to the negative control group (vehicle).

This result suggests that the fusion peptide 4 of the present disclosure can increase autophagy in tissues suffering from atherosclerosis and alleviate atherosclerosis induced by a high-cholesterol diet.

### CONCLUSION

The fusion peptide consisting of the amino acid sequence represented by SEQ ID NO: 4 of the present disclosure may help prevent or at least delay the progression of a disease related to autophagy disorder induced by reduced intracellular autophagy by inhibiting the interaction of the endoplasmic reticulum and mitochondria and/or PPI between IP₃R and GRP75, thereby increasing the distance between the endoplasmic reticulum and mitochondria, thereby inhibiting calcium transfer from the endoplasmic reticulum to mitochondria, and increasing lysosomal activity and autophagic activity.

Although the specific exemplary embodiments of the present disclosure have been described in detail above, it is obvious for those having knowledge in the art that they are merely specific exemplary embodiments and, therefore, the scope of the present disclosure is not limited by them. Accordingly, the substantial scope of the present disclosure should be defined by the attached claims and their equivalents.

### [Sequence Text List]

Attached sequence list e-file (C:\Users\ [symbol] \Desktop\HP11732.xml)

What is claimed is:

1. A method for treating a disease related to autophagy disorder, the method comprising inhibiting protein-protein interaction (PPI) between IP₃R (inositol 1,4,5-trisphosphate (IP₃) receptor) and GRP75 (glucose-related protein 75) in MAMs (mitochondria-associated membranes) in cells of a subject in need thereof.

2. The method according to claim 1, wherein an inhibitor of the protein-protein interaction (PPI) between IP₃R (inositol 1,4,5-trisphosphate (IP₃) receptor) and GRP75 (glucose-related protein 75) in MAMs (mitochondria-associated membranes) is administered to the subject.

3. The method according to claim 2, wherein the inhibitor inhibits the interaction between the endoplasmic reticulum and mitochondria in cells.

4. The method according to claim 3, wherein the inhibitor is a fusion peptide for inhibiting protein-protein interaction (PPI) between IP₃R (inositol 1,4,5-trisphosphate (IP₃) receptor) and GRP75 (glucose-related protein 75) in MAMs (mitochondria-associated membranes), which contains a peptide represented by the amino acid sequence of SEQ ID NO: 4 and a cell-penetrating peptide (CPP); a nucleic acid molecule encoding the fusion peptide; an expression vector comprising the nucleic acid molecule; or a host cell transformed with the expression vector.

5. The method according to claim 4, wherein the cell-penetrating peptide is represented by any amino acid sequence selected from SEQ ID NOS: 5 to 20.

6. The method according to claim 4, wherein the fusion peptide is represented by any amino acid sequence selected from SEQ ID NO: 24 and SEQ ID NOS: 30 to 44.

7. The method according to claim 1, wherein the disease related to autophagy disorder is a disease caused by abnormal protein accumulation induced by decreased autophagy in cells or a degenerative disease.

8. The method according to claim 7, wherein the disease caused by abnormal protein accumulation or the degenerative disease is any one selected from arteriosclerosis, pulmonary hypertension, Alzheimer's disease, Parkinson's disease, type 2 diabetes, amyotrophic lateral sclerosis, dialysis-related amyloidosis, cystic fibrosis, sickle cell anemia, Huntington's disease, Creutzfeldt-Jakob disease, Lewy body dementia, inclusion body myositis, cerebral amyloid angiopathy, traumatic brain injury, frontotemporal dementia, progressive supranuclear palsy, corticobasal degeneration, Pick's disease, and argyrophilic grain disease.

9. The method according to claim 8, wherein the arteriosclerosis is atherosclerosis.

10. The method according to claim 9, wherein the inhibitor reduces intracellular lipids by inducing autophagy in atherosclerosis.

11. A fusion peptide for inhibiting protein-protein interaction (PPI) between IP₃R (inositol 1,4,5-trisphosphate (IP₃) receptor) and GRP75 (glucose-related protein 75) in MAMs (mitochondria-associated membranes), which comprises a peptide represented by the amino acid sequence of SEQ ID NO: 4 and a cell-penetrating peptide (CPP).

12. The fusion peptide according to claim 11, wherein the cell-penetrating peptide is represented by any amino acid sequence selected from SEQ ID NOS: 5 to 20.

13. The fusion peptide according to claim 11, wherein the fusion peptide is represented by any amino acid sequence selected from SEQ ID NO: 24 and SEQ ID NOS: 30 to 44.

14. A pharmaceutical composition comprising a fusion peptide for inhibiting protein-protein interaction (PPI) between IP₃R (inositol 1,4,5-trisphosphate (IP₃) receptor) and GRP75 (glucose-related protein 75) in MAMs (mitochondria-associated membranes), which comprises a peptide represented by the amino acid sequence of SEQ ID NO: 4 and a cell-penetrating peptide (CPP); a nucleic acid molecule encoding the fusion peptide; an expression vector comprising the nucleic acid molecule; or a host cell transformed with the expression vector, as an active ingredient.

15. The pharmaceutical composition according to claim 14, wherein the cell-penetrating peptide is represented by any amino acid sequence selected from SEQ ID NOS: 5 to 20.

16. The pharmaceutical composition according to claim 14, wherein the fusion peptide is represented by any amino acid sequence selected from SEQ ID NO: 24 and SEQ ID NOS: 30 to 44.

17. The pharmaceutical composition according to claim 14, wherein the pharmaceutical composition is for treating a disease related to autophagy disorder.

18. The pharmaceutical composition according to claim 17, wherein the disease related to autophagy disorder is a disease caused by abnormal protein accumulation induced by decreased autophagy in cells, or a degenerative disease.

19. The pharmaceutical composition according to claim 18, wherein the disease caused by abnormal protein accumulation or the degenerative disease is any one selected from arteriosclerosis, pulmonary hypertension, Alzheimer's disease, Parkinson's disease, type 2 diabetes, amyotrophic lateral sclerosis, dialysis-related amyloidosis, cystic fibrosis, sickle cell anemia, Huntington's disease, Creutzfeldt-Jakob disease, Lewy body dementia, inclusion body myositis, cerebral amyloid angiopathy, traumatic brain injury, frontotemporal dementia, progressive supranuclear palsy, corticobasal degeneration, Pick's disease, and argyrophilic grain disease.
